(19) United States
(12) Patent Application Publication
(10) Pub. No.: US 2010/0003232 A1
(43) Pub. Date: Jan. 7, 2010

(54) INSULIN-DEGRADING ENZYME MUTANTS AND METHODS OF USE

(76) Inventors: Wei-jen Tang, Chicago, IL (US); Yuequan Shen, Tianjin (CN)

Correspondence Address: MICHAEL BEST & FRIEDRICH LLP, ONE SOUTH PINCKNEY STREET, P O BOX 1806, MADISON, WI, 53701, US

(21) Appl. No.: 12/442,198

(22) PCT Filed: Sep. 24, 2007
(86) PCT No.: PCT/US07/79297

§ 371 Date: Mar. 20, 2009

Related U.S. Application Data
(60) Provisional application No. 60/826,676, filed on Sep. 22, 2006.
(60) Provisional application No. 60/888,140, filed on Feb. 5, 2007.

(51) Int. Cl. A61K 38/44; C12N 9/02; C12N 5/02; C12N 15/63; A61K 31/7088; C07H 21/04

(52) U.S. Cl. 424/94.4; 435/189; 435/325; 435/320.1; 514/44 R; 536/23.2

(57) ABSTRACT

Disclosed are mutant polypeptides of insulin degrading enzymes having at least 95% amino acid identity to SEQ ID NO: 1, having at least one mutation in a region corresponding to human IDE-N or human IDE-C, having increased activity, polynucleotides encoding the polypeptides, and methods of use.

### CROSS-REFERENCE TO RELATED APPLICATIONS

This application claims priority to U.S. Provisional Application No. 60/826,676 filed Sep. 22, 2006 and U.S. Provisional Application No. 60/888,140 filed Feb. 5, 2007, each of which is incorporated by reference in its entirety.

### STATEMENT REGARDING FEDERALLY SPONSORED RESEARCH

This invention was supported in part by National Institutes of Health grants R01GM81539, R01GM62548, and 5P60DK020595-30 funded pilot grant. The United States government has certain rights in this invention.

### INTRODUCTION

Insulin-degrading enzyme (IDE) is a Zn²⁺-metalloprotease that catalyzes the proteolysis of several substrates, including insulin, glucagon, amylin, and amyloid β (Aβ). Loss-of-function mutations of IDE in rodents cause glucose intolerance and cerebral Aβ accumulation, whereas enhanced IDE activity effectively reduces brain Aβ. Thus, IDE is relevant to various diseases, including diabetes, insulin resistance, and Alzheimer's disease. There is a need in the art for improved understanding of the interaction between IDE and its substrates to facilitate development of compositions and methods for modulating IDE activity.

### SUMMARY OF THE INVENTION

In one aspect, the present invention provides a mutant polypeptide of insulin degrading enzyme having increased activity relative to that of SEQ ID NO:1. The mutant polypeptide has at least 95% amino acid identity to SEQ ID NO:1 and has at least one mutation in a region corresponding to human IDE-N or human IDE-C.

In another aspect, the present invention provides a mutant polypeptide of insulin degrading enzyme having reduced oligomerization relative to oligomerization of the insulin degrading enzyme of SEQ ID NO:1.

Also provided is a polynucleotide encoding the polypeptide of the invention.

The present invention provides cells comprising the polynucleotides of the invention.

In yet another aspect, the invention provides an insulin degrading enzyme chemically modified to have reduced interaction between IDE-N and IDE-C, relative to a corresponding polypeptide that is not chemically modified.

The present invention also provides a method of reducing amyloid β or insulin in a subject comprising administering the polynucleotide or polypeptide of the invention to the subject in an amount effective to reduce amyloid β or insulin.

Also provided are methods of reducing Aβ comprising contacting a cell expressing Aβ with a polypeptide of the invention.

### BRIEF DESCRIPTION OF THE DRAWINGS

FIG. 1 shows a phylogenic tree of insulin degrading enzyme and compares the percent similarity and identity of various insulin degrading enzyme homologs to human insulin degrading enzyme.

FIG. 2 provides a sequence alignment of IDE interacting peptides.

FIG. 3 is a representation of the structure of IDE-E111Q complexed with insulin B chain.

FIG. 4 depicts the interaction between IDE and insulin B chain, Aβ(1-40), amylin, and glucagon.

FIG. 5A-D show the amino acid sequence alignment of human IDE (SEQ ID NO:1) domains 1-4 with homologs from fruitfly (SEQ ID NO:2), zebrafish (SEQ ID NO:3), cress (SEQ ID NO:4), yeast (SEQ ID NO:5), and nematode (SEQ ID NO:6); amino acid residues participating in substrate binding or located at the interface between IDE-N and IDE-C are denoted with an “S” or “I” below the residue.

FIG. 6 depicts the substrate binding chamber of IDE.

FIG. 7 depicts the interaction between Aβ and IDE domain 4 residues Y831 and R824 and the activity of IDE mutants relative to wild type.

FIG. 8 shows the activities of IDE mutants having a single mutation in domain 4 or in the catalytic base in domain 1 (FIG. 8A), and of double cysteine mutants of IDE mutants (C) or wild type IDE in the presence and absence of reducing agent or oxidizing agent.

FIG. 9 shows the conformational changes of IDE substrates and their cleavage sites.

FIG. 10 depicts oligomerization of IDE molecules (FIG. 10A), and atoms that interact between IDE molecules (FIG. 10B) or IDE dimers (FIG. 10C) to promote oligomerization.

### DETAILED DESCRIPTION OF THE INVENTION

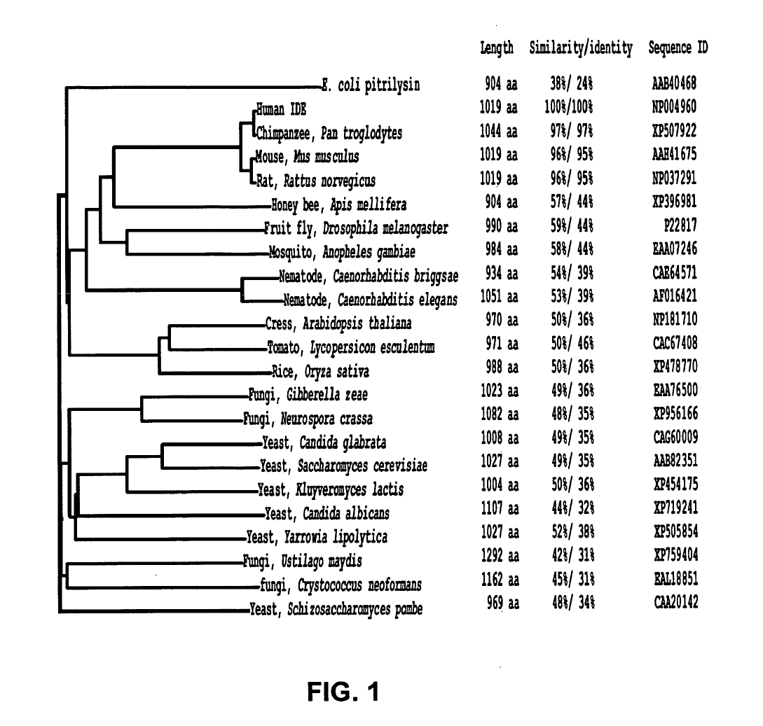

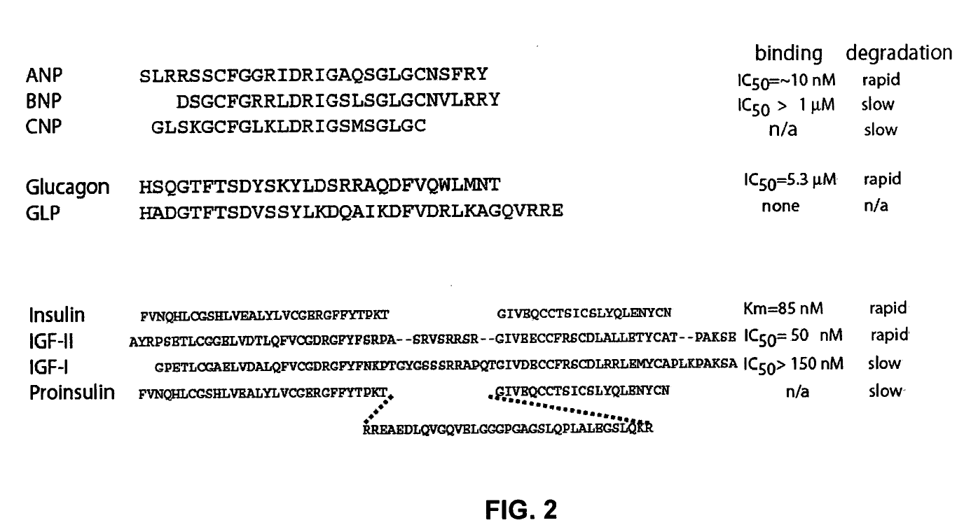

IDE, originally identified by its ability to rapidly degrade insulin, is a highly conserved zinc metalloprotease found in bacteria, fungi, plants, and animals (FIG. 1). IDE is unusual for its high affinity to its substrates, which are highly diverse in sequence and structure. Furthermore, IDE is remarkable for its capacity to selectively cleave certain hormones without degrading related family members (FIG. 2). IDE cleaves its substrates multiple times at cleavages sites having no obvious recognition motifs. The molecular basis by which IDE exhibits high selectivity but degenerate cleavage sites for a broad range of hormones has remained elusive.

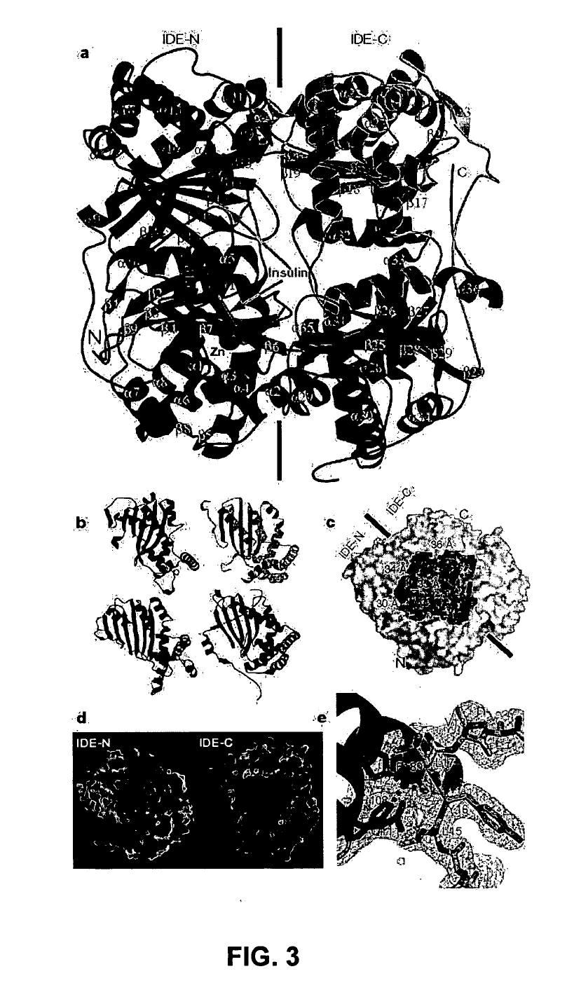

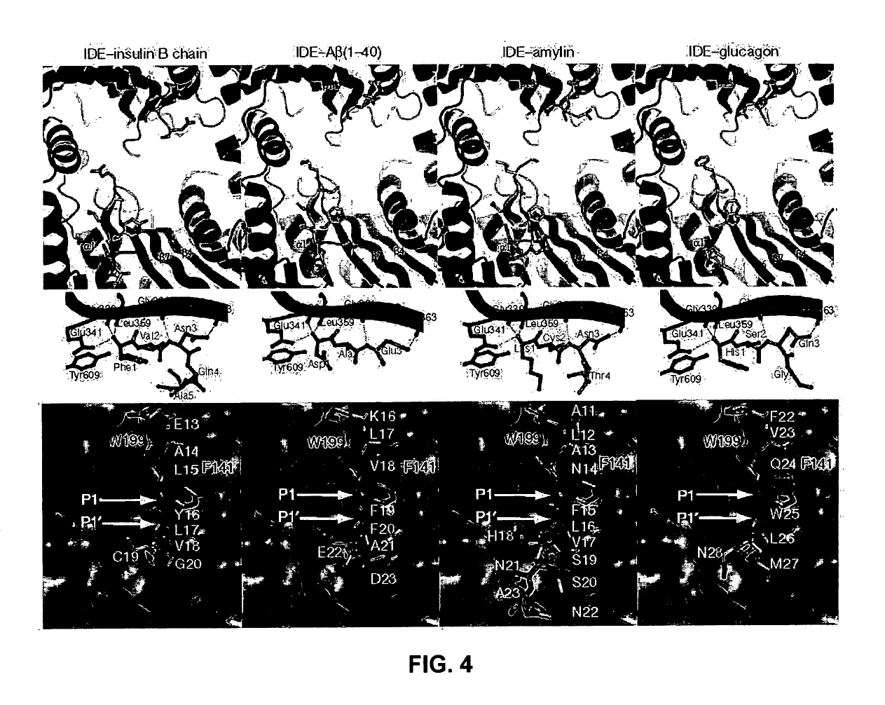

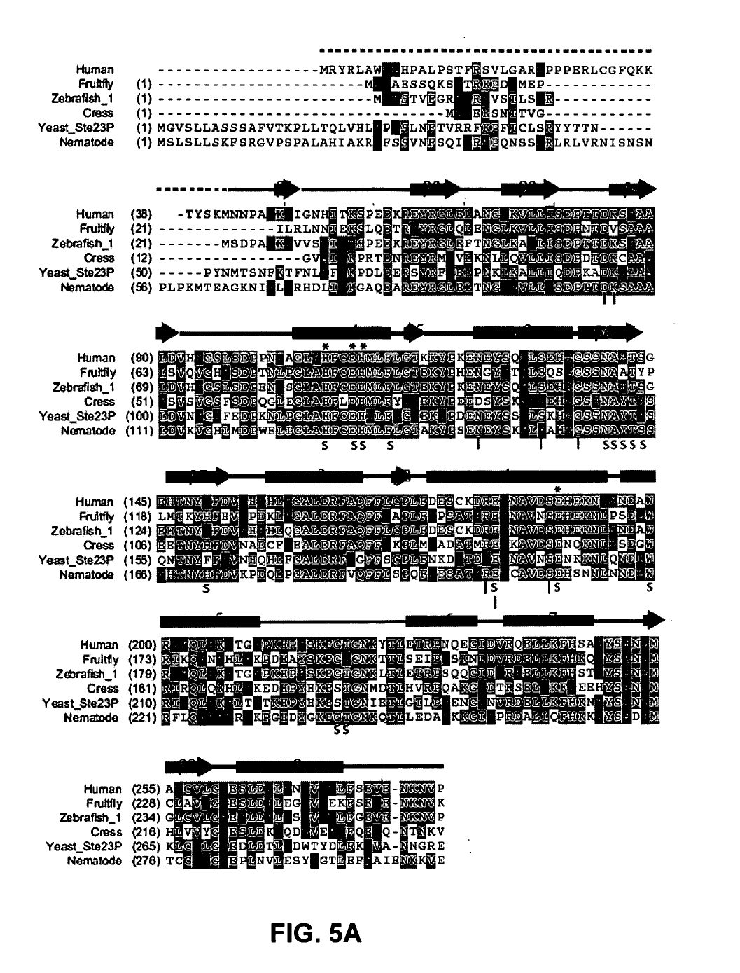

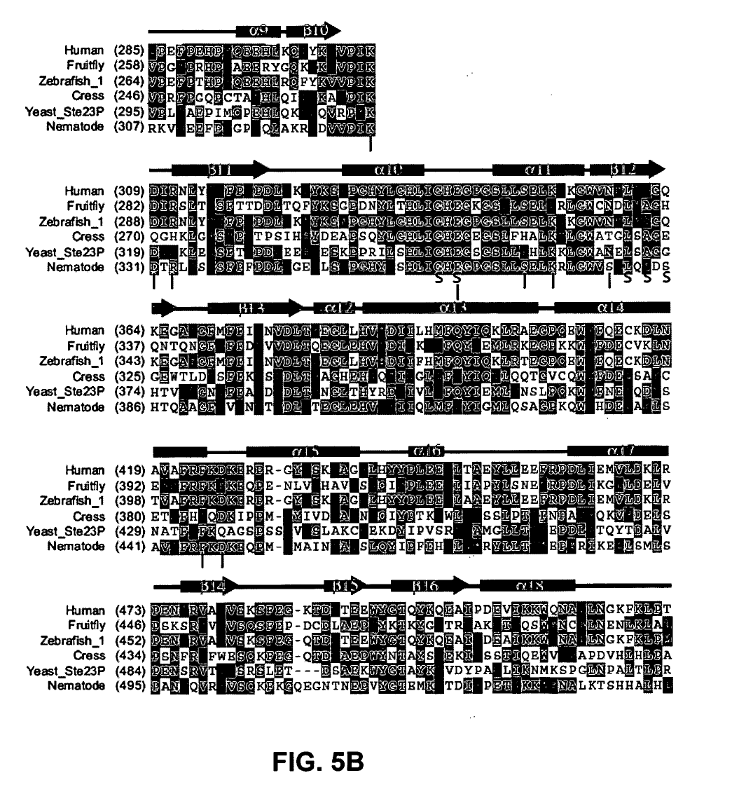

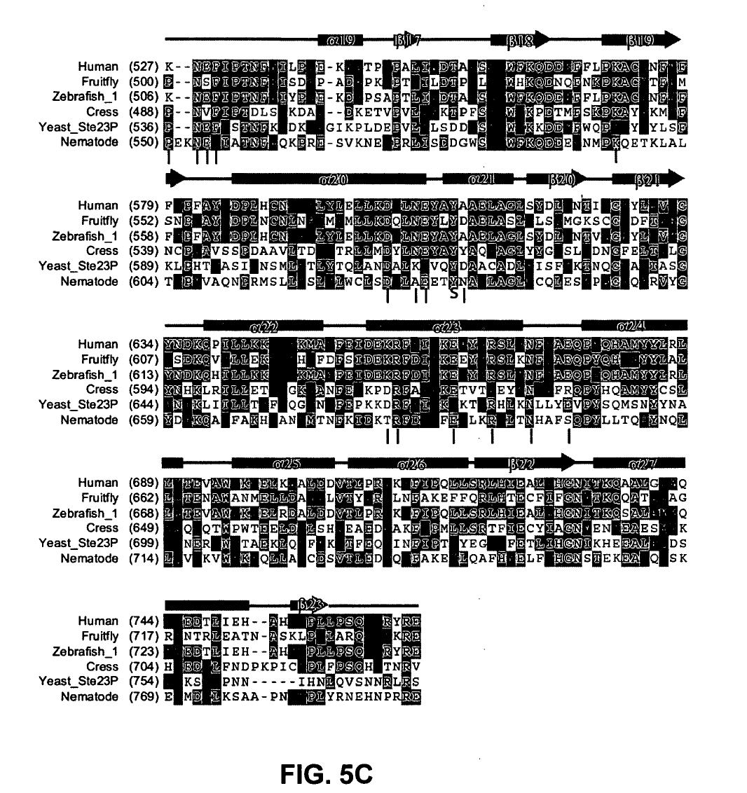

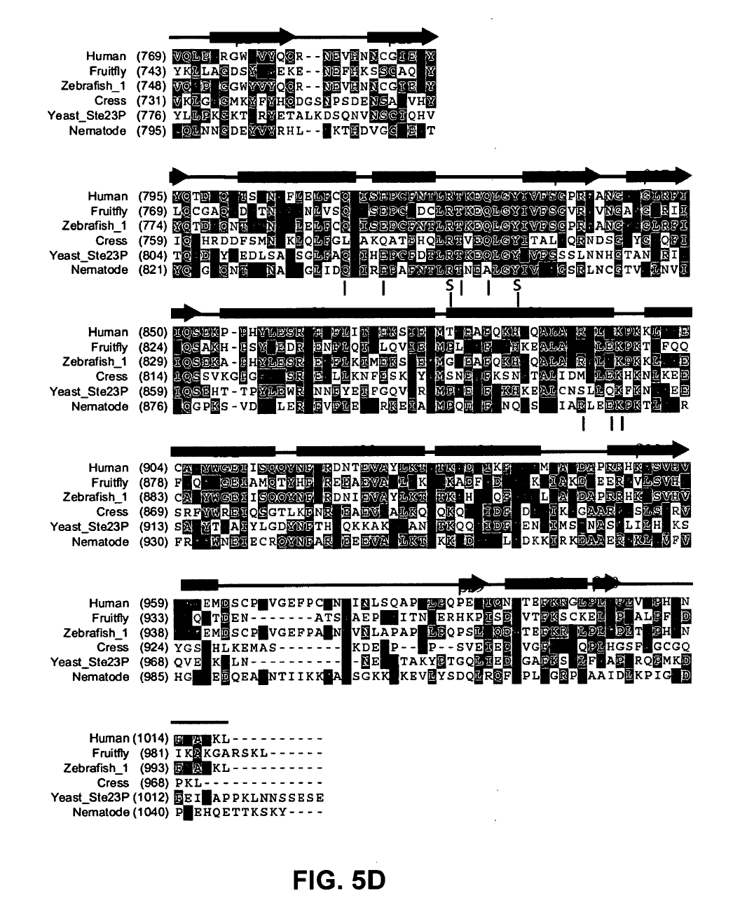

To understand substrate recognition and catalytic mechanism of IDE, the structures of human IDE in complex with four substrates (insulin B chain, Aβ(1-40), amylin, and glucagon) were solved, as described below. The crystal structures of the 113 kDa human, Zn²⁺-bound, catalytically inactive IDE-E111Q in complex with insulin B chain was solved at 2.25 Å resolution (FIG. 3A) and the crystal structures of Zn²⁺-free IDE-E111Q in complex with Aβ(1-40), amylin, and glucagon was solved at 2.1 Å, 2.6 Å, and 2.5 Å resolution, respectively (FIG. 4). Each IDE monomer is comprised of four structurally homologous αβ roll domains (domain 1, aa 43-285; domain 2, aa 286-515; domain 3, aa 542-768; and domain 4, aa 769-1016) (FIG. 3B; FIG. 5A-D) that share less than 25% sequence similarity. The N-terminal domains 1 and 2 (IDE-N) form a αβαβα sandwich, as do C-terminal domains 3 and 4 (IDE-C).

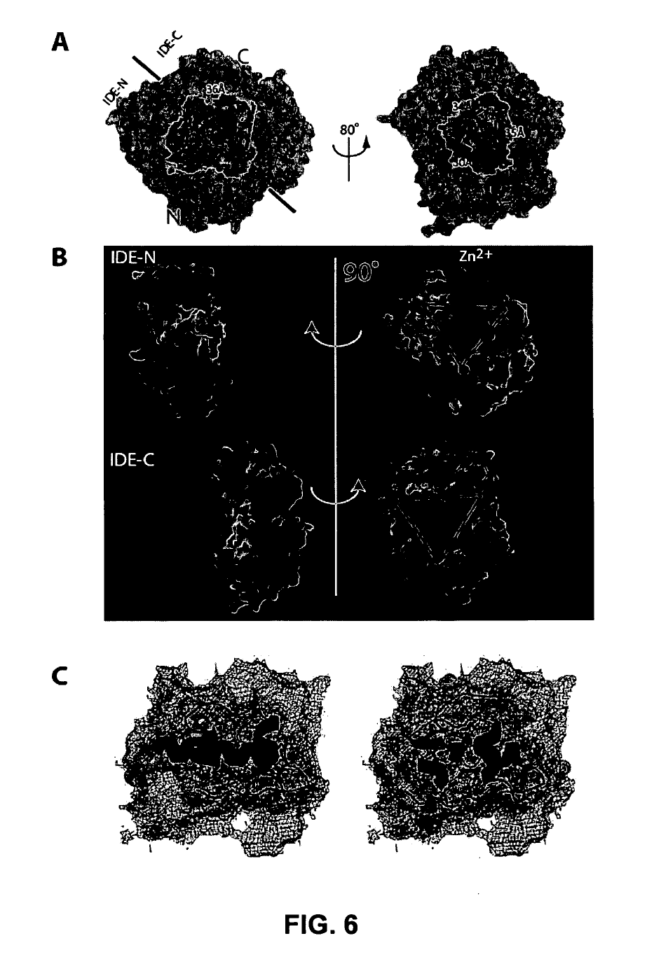

IDE-N and IDE-C are joined by a 28-aa extended loop and form an enclosed chamber or cavity, shaped like a triangular prism, with triangular base dimensions of 35 Å by 34 Å by 30 Å and a height of 36 Å. This enclosed cavity has a total volume of ˜1.3×10⁴ Å³, just large enough to encapsulate insulin AB chains (FIG. 3C; FIG. 6). All four domains contribute surface to the internal chamber. The surface provided by IDE-N is largely neutral or negatively charged; however, that from IDE-C is predominantly positively charged (FIG. 3D). IDE domain 1 contains the catalytic site with a zinc ion coordinated by two histidines (aa 108 and aa 112) and one glutamate (aa 189) (FIG. 3A, FIG. 3E).

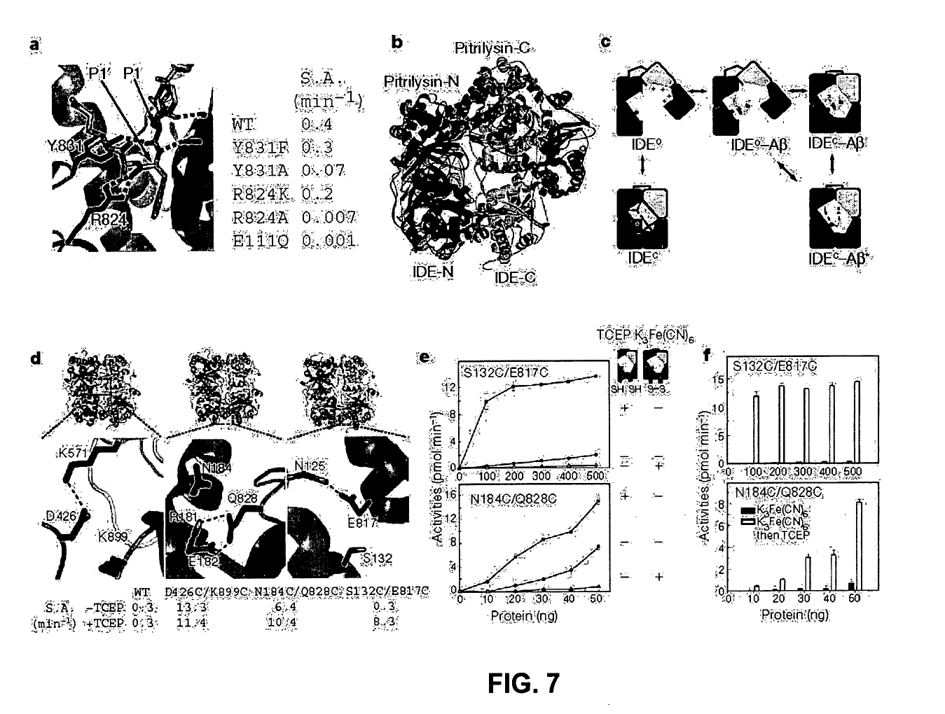

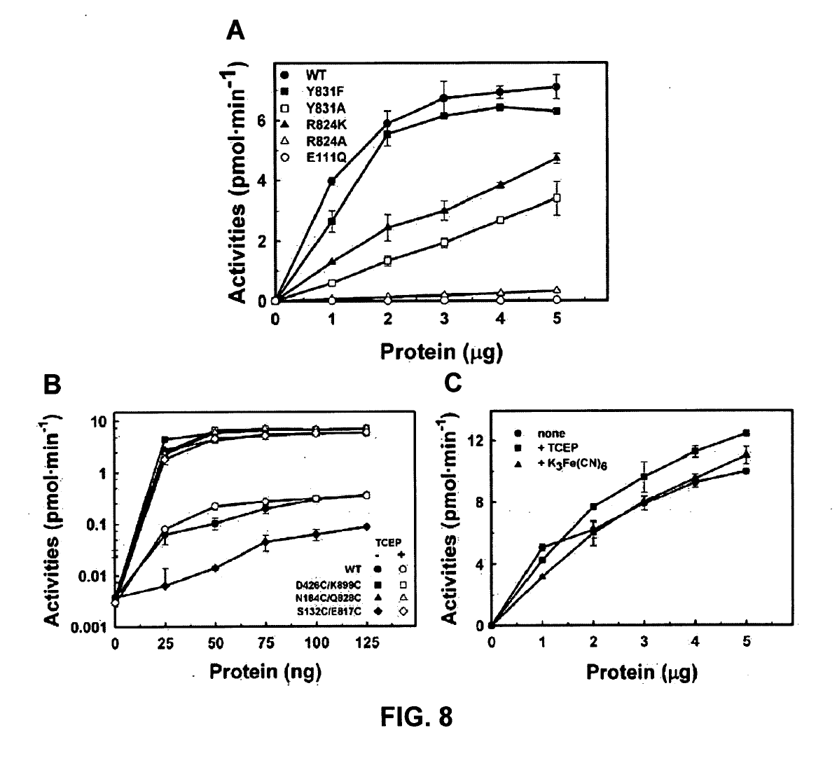

Two discrete segments of four sequence diverse substrates, insulin B chain, Aβ(1-40), amylin, and glucagon, are clearly visible in structures of the IDE-substrate complex, and they share similar features (FIG. 4). The N-terminal 3-5 amino acids and cleavage site-containing 7-13 amino acids of all four substrates form β-sheets with IDE β12 and β6 strands, respectively. The remaining regions (55-72%) of all four substrates in the IDE-substrate complexes are disordered, although they are present in the chamber, as verified by mass spectrometry analysis. At the catalytic site, multiple residues of IDE domain 1 and 4 form a largely polar cavity with patches of hydrophobic and charged regions that interact with cleavage sites in all four substrates. The bulky hydrophobic residues at the P1 sites of the IDE substrates interact with Phe141 at the S1 site of IDE domain 1, while the hydrophobic residues of the P1′ sites are buried deeply in the hydrophobic patch surrounding S1′ of IDE domain 1. In addition, Arg 824 and Tyr 831 of IDE domain 4 form hydrogen bonds with the P1 and P1′ sites of substrates (FIG. 7A). Mutations of these two residues to alanine substantially reduce the catalytic rate of IDE (FIG. 7A, FIG. 8A). This is consistent with IDE-N serving as the catalytic domain while IDE-C facilitates the binding of substrates.

The chamber or cavity formed by interaction between IDE-N and IDE-C serves as an enclosed substrate-binding compartment that prevents the entry and exit of substrates. Thus, IDE needs to undergo a significant conformational change from the open state, which can accept substrate, to the closed state for proper substrate recognition and catalysis. A structural comparison of substrate-bound IDE with substrate-free E. coli pitrilysin (accession code=1Q2L) reveals how repositioning between IDE-N and IDE-C can lead to the open state, which allows substrate access to catalytic cavity (FIG. 7B). Pitrilysin shares 25% sequence identity with IDE and is arranged as two globular entities, pitrilysin-N and pitrilysin-C (FIG. 7B). Structural comparison of pitrilysin and IDE reveals that pitrilysin-C rotates 54° away from pitrilysin-N so that domain 4 of pitrilysin does not contact domain 1. Thus, IDE may normally equilibrate between the substrate-free open state (IDE^{O}) and closed state (IDE^{C}) (FIG. 7C). IDE^{C} cannot bind substrates but it is capable of degrading substrate after substrates are entrapped inside the catalytic chamber. IDE^{O} can bind substrates; however, without residues from IDE-C (i.e. Arg824 and Tyr831 of domain 4) to bind substrates, IDE^{O} is less active than IDE^{C}. After the transition from IDE^{O} to IDE^{C}, the entrapped peptides need to fit into the catalytic cleft of IDE to enable their cleavage one or multiple times before the reopening of IDE.

The crystal structures reveal that IDE-N and IDE-C have extensive interactions that bury a large surface (11,496 Å²) with good shape complementarity (Sc value=0.66) and numerous hydrogen bonds (Table 1). For this reason, we hypothesized that, in the absence of interactions with other proteins or factors, the substrate-free IDE^{C} state is stable and the catalytic chamber of IDE is mostly closed. To test this hypothesis, we constructed three IDE mutants (D426C/K899C, N184C/Q828C, S132C/E817C), each having double cysteine mutations that loosen the contacts between IDE-N and IDE-C, thereby promoting the opening of the catalytic chamber and increasing the catalytic rate (FIG. 7D). The two cysteine mutations were designed to potentially form a disulfide bond, thus permitting the mutants to be locked in the closed conformation.

TABLE 1
List of atoms from human IDE-N and IDE-C that are in close contact
with each other and the measured distance between them in the crystal
structure.

| Atoms from IDE-N | Atoms from IDE-C | Distance (Å) |
|---|---|---|
| [ASP 84 OD2] | [LYS 896 N] | 3.1 |
| [LYS 85 NZ] | [ASP 895 OD2] | 3.3 |
| [ASN 125 OD1] | [GLU 817 OE1] | 3.1 |
| [SER 132 O] | [GLN 813 NE2] | 3.2 |
| [SER 132 O] | [ARG 892 NH2] | 3.3 |
| [GLY 136 O] | [ARG 892 NH1] | 2.6 |
| [ARG 181 NH1] | [THR 825 O] | 2.8 |
| [GLU 182 OE1] | [ARG 824 NH2] | 3.2 |
| [GLU 182 OE2] | [GLN 828 OE1] | 3.3 |
| [ALA 185 N] | [GLN 828 NE2] | 3.1 |
| [SER 188 OG] | [TYR 831 N] | 2.7 |
| [LYS 308 NZ] | [GLU 676 OE1] | 2.8 |
| [ASP 309 O] | [ARG 668 NH1] | 2.8 |
| [ASP 309 N] | [ASN 672 ND2] | 2.8 |
| [ARG 311 NH2] | [GLU 664 OE2] | 2.5 |
| [ARG 311 NH2] | [ARG 668 NE] | 3.1 |
| [GLU 341 OE2] | [ASN 605 OD1] | 2.9 |
| [SER 348 OG] | [GLU 606 OE2] | 2.7 |
| [LYS 351 NZ] | [ASP 602 OD2] | 2.9 |
| [LYS 351 O] | [LYS 657 NZ] | 2.9 |
| [ASN 357 OD1] | [ARG 658 NH2] | 3.0 |
| [PHE 424 O] | [LYS 571 NZ] | 2.8 |
| [ASP 426 OD1] | [LYS 571 NZ] | 2.9 |
| [LYS 527 O] | [GLU 529 N] | 3.1 |
| [ASN 528 OD1] | [PHE 530 N] | 2.9 |
| [ASN 528 ND2] | [ALA 610 O] | 2.9 |

When fluorogenic substrate V was used as a substrate, all three IDE double cysteine mutants, D426C/K899C, N184C/Q828C, S132C/E817C, had 30-40 fold higher catalytic activity than wild type IDE in the presence of the reducing agent TCEP (FIG. 7E). The elevated proteolytic activities of these three IDE mutants were confirmed when either insulin or the amyloid-β peptide (1-42) were used. The IDE mutants were evaluated for inactivation in the presence of K₃Fe(CN)₆, an oxidizing agent, which facilitates disulfide bond formation. Both S132C/E817C and N184C/Q828C were found to have reduced activity in the presence of K₃Fe(CN)₆ (FIG. 7E-F). The activities of S132C/E817C and N184C/Q828C were restored when TCEP, a reducing agent, was added (FIG. 7F). The third mutant, D426C/K899C, was not inactivated by K₃Fe(CN)₆ presumably due to a failure to form a disulfide bond between the cysteine residues at amino acid positions 426 and 899. Wild type IDE was included as a control and its activity was found to be insensitive to treatment with TCEP or K₃Fe(CN)₆ (FIG. 7D; FIG. 8C) The oligomeric state of these three IDE mutants is similar to that of wild type IDE, excluding a difference in oligomerization as the cause for their increased activity.

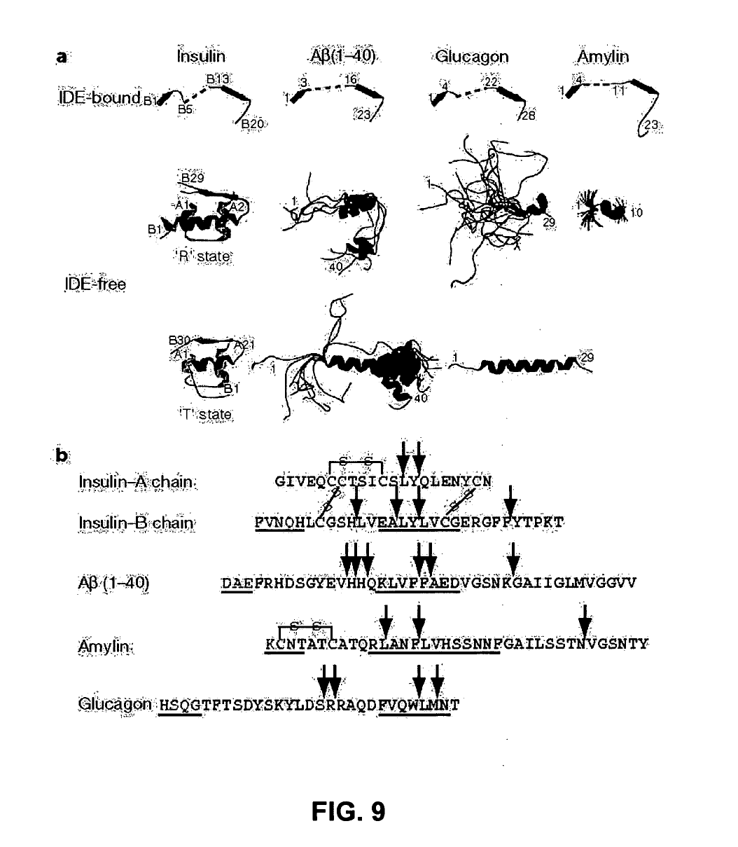

The comparison of IDE-free and IDE-bound insulin B chain, Aβ(1-40), amylin and glucagon reveals substantial conformational changes of IDE substrates upon binding to IDE (FIG. 9A). Both the N-terminal loop and the α-helical cleavage site turn into β-strands. IDE cleaves insulin B chain and Aβ at multiple sites. The binding of insulin B chain and Aβ(1-40) to the IDE catalytic cleft positions both substrates for cleavage at known sites. IDE also cleaves amylin and glucagon at multiple locations not been previously identified (FIG. 9B). MALDI-TOF analysis shows that the cleavage sites for amylin and glucagon correspond to degradation sites depicted by the crystal structures of the IDE-substrate complex.

Structural and biochemical analyses reveal that at least four factors contribute to the unique mechanism of substrate recognition by IDE. Favorable binding of the substrate N-terminus and cleavage sites to β-strands within IDE and proper anchoring of the cleavage site within the catalytic cleft are clearly key specific determinants. In addition, peptides that do not have significant positive charges at the C-terminus and avoid the charge repulsion from IDE-C are better IDE substrates than substrates lacking these features. The IDE-substrate structures show that the C-termini of insulin B chain, Aβ, and amylin make substantial contacts with the IDE inner cavity, which is highly positively charged. BNP, glucagon-like peptide, and IGF-I, which have multiple positively charged residues at their C-termini, are poor substrates. However, the related hormones, ANP, glucagon, and IGF-II, which lack positive charges at their C-termini, are excellent IDE substrates. The fourth determining factor is size. The catalytic chamber of IDE is large enough to accommodate only relatively small peptides (estimated to be less than 50-aa long). Larger peptides such as TGF-β and pro-insulin are less likely to be entrapped by IDE than the related, smaller hormones, TGF-α and insulin. Consequently, the degradation of such larger peptides is significantly slower.

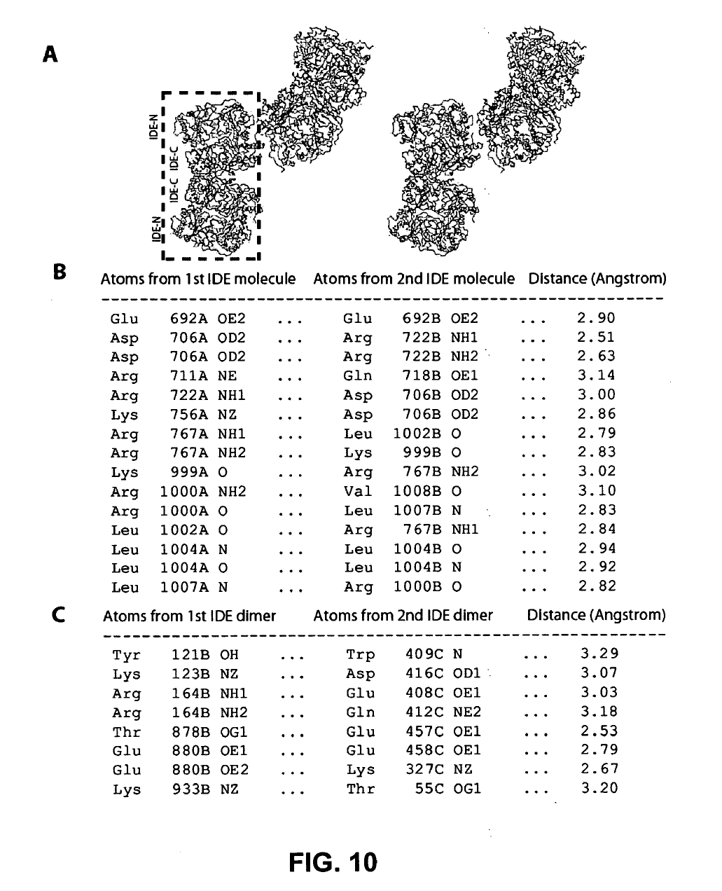

IDE, an M16A member of the zinc metalloprotease family, shares similar secondary structure and domain organization with yeast mitochondria processing peptidase (MPP), a distally related M16B member. Similar to IDE, MPP also use the exosite for substrate recognition. However, the catalytic chamber of MPP stays open, whereas IDE has a buried catalytic site within the structure and access to this chamber is kinetically controlled by the closed-open conformational switch. IDE can also self-oligomerize, and interaction between two IDE dimers could lock IDE in the IDE^{C} state (FIG. 10), which may explain how oligomerization allosterically regulates the catalytic activity of IDE. Small molecules that could shift the equilibrium between IDE^{C} and IDE^{O} toward the open state or reduce IDE oligomerization will likely allosterically regulate the activity of IDE. Such compounds might facilitate the clearance of amyloid-β and other pathologically relevant IDE substrates.

It is specifically envisioned that mutants can be used to treat, prevent or ameliorate conditions associated with pathologically relevant IDE substrates, including, for example, insulin resistance, Type II Diabetes, and Alzheimer's Disease. It is envisioned that IDE mutants having enhanced activity would be particularly useful.

As used herein, an IDE mutant having enhanced activity or increased activity is one in which its ability to cleave at least one IDE substrate, whether a natural substrate or artificial substrate, is enhanced relative to the ability of wild type human IDE (SEQ ID NO:1) to cleave the same substrate under like conditions. Any suitable assay may be used, including those described herein. Preferably, the activity of the mutant is increased at least 10%. More preferably, the activity of the mutant is increased 25%, at least 50%, at least 100% or more. More preferably still, the activity of the mutant is increased at least 2 fold, 5 fold, 10 fold, 20 fold, 30 fold, 40 fold, or more.

As described in the Examples, three IDE double cysteine mutants into which a cysteine residue was introduced into each of IDE-N and IDE-C (D426C/K899C, N184C/Q828C, S132C/E817C) had 30-40 fold higher catalytic activity in the presence of a reducing agent. It is expected that other residues within IDE-N or IDE-C could be replaced with a cysteine residue to alter or disrupt the interaction between IDE-N and IDE-C to open the catalytic chamber and increase the catalytic rate. If two residues are replaced, one in each of IDE-N and IDE-C, the activity of the mutant may be altered by altering the reducing conditions in the environment of the enzyme. For example, the activity could be reduced under oxidizing conditions and increased under reducing conditions.

In addition, one of skill in the art could readily develop IDE mutants having altered catalytic activity in which interactions between amino acid residues of the IDE-N and IDE-C domains are disrupted. For example, interaction between IDE-N and IDE-C could be reduced by constructing mutants in which one or more amino acid residues at the interface between IDE-N and IDE-C is replaced with an amino acid residue with reduced propensity to form a salt bridge or hydrogen bond with its opposing amino acid residue. Candidate residues include those identified as appearing at the interface (FIG. 5; Table 1). It is envisioned that human IDE mutants having a mutation in at least one member of at least one of the amino acid pairs listed in Table 1, or within five amino acid residues of at least one member of at least one of the amino acid pairs listed in Table 1, will disrupt the interaction between the amino acid pairs. Such mutants will likely have enhanced activity, relative to wild-type.

For example, LYS85, GLU182, LYS308, ARG311, LYS351, and ASP426 of IDE-N form salt bridges with ASP895, ARG824, GLU676, GLU664, ASP602, and LYS571 of IDE-C, respectively. By replacing one or more members of the salt bridge pair with another amino acid unable to form a salt bridge, the interaction between IDE-N and IDE-C would be weakened such that the catalytic activity of the enzyme would be increased. The remaining amino acid pairs listed in Table 1 are believed to interact through hydrogen bonding. Hydrogen bonding between pair members may be disrupted by replacing one or more pair members with an amino acid unable to participate in hydrogen bonding. It is envisioned that small, neutral amino acids such as alanine, glycine, leucine, and isoleucine will be particularly suitable for use in replacing the amino acid residues that natively participate in the interaction between the IDE-N and IDE-C domains.

Additionally, IDE mutants having increased catalytic activity may be developed by introducing mutations that exhibit reduced oligomerization and form dimers and tetramers less frequently than do wild type IDE molecules. It is specifically envisioned that mutants having a mutation in an amino acid that participates in dimerization or tetramerization will have reduced dimer and tetramer formation and thus increased activity. FIG. 10 lists pairs of amino acids that interact between two IDE molecules (FIG. 10B) or between two IDE dimers (FIG. 10C) to promote dimerization or tetramerization. A mutation in one or more of these amino acid residues is likely to produce a mutant having reduced dimerization and increased activity.

The present invention provides extensive information provided concerning the secondary structure of human, and which amino acids are important to its function. This information makes it possible for one skilled in the art to design mutant polypeptides having enhanced activity. Suitably, the mutants have at least 85% amino acid identity to SEQ ID NO:1. Preferably, the mutants have at least 90% amino acid identity to SEQ ID NO:1, at least 93% amino acid identity to SEQ ID NO:1, at least 95% amino acid identity to SEQ ID NO:1, or at least 97% amino acid identity to SEQ ID NO:1. As used herein, “percent identity” or “% identity” of a mutant of IDE is determined by comparing the whole of SEQ ID NO:1 to the sequence of the mutant using a computer implemented algorithm, specifically, the algorithm of Karlin and Altschul (Proc. Natl. Acad. Sci. 87: 2264-68 (1990), modified Proc. Natl. Acad. Sci. 90: 5873-77 (1993)), using the default parameters.

As an alternative to or as a supplement to developing IDE mutants with reduced interaction between IDE-N and IDE-C, or between IDE molecules or dimers, suitably one could obtain modified IDE proteins having increased activity according to the invention by chemically modifying the protein. Chemical modifications may be added to the polypeptide by reacting a chemical with a functional group on an amino acid residue of the protein, such as an amine, carboxyl, thiol or hydroxyl group. See Chen et al., (2005) Chem Biol, 12: 317-383 and Kochendoerfer et al., (2003) Science, 299: 884-887, each of which is incorporated herein by reference in its entirety. Chemicals useful in making such modifications include, but are not limited to, polymers like polyethylene glycol (PEG), polypeptides such as the Fc portion of an antibody or chemical groups. Chemical modification of the IDE protein at any of the amino acids at the interface of IDE-C and IDE-N may be used to alter the interaction of IDE-C and IDE-N and result in increased catalytic activity of IDE. For example, chemical modifications, e.g., addition of a polymer, to one or more of the amino acids listed in Supplemental FIG. 4E may interrupt the hydrogen bonding interactions and salt bridge formation between the amino acids leading to increased activity.

Chemical modifications, such as addition of polymers, may also be added to mutated amino acids residues within the protein. For example, the IDE double cysteine mutants described in the Examples could be PEGylated by reaction with thiol-reactive PEGs. One of skill in the art would expect that these proteins would behave similarly to the double cysteine mutant proteins after treatment with a reducing agent and have increased activity irrespective of the presence or absence of oxidizing or reducing agents. The resulting proteins may contain multiple PEGs. The amount of PEG additions could be chemically controlled or proteins containing only one of the described cysteine mutations could be used. Notably, all of the cysteine residues in IDE can be mutated leaving only those cysteines in the IDE-N and IDE-C interaction region. Thus, chemical modification by reaction through the thiol of cysteines would be less likely to result in a large number of chemical modifications within IDE.

In addition, chemical modifications, such as polymer conjugation or addition of an Fc polypeptide to proteins, may increase protein solubility and stability. Polymer conjugation has been shown to also reduce protein immunogenicity and prolong the plasma half-life of proteins through prevention of renal elimination and avoidance of receptor-mediated protein uptake by cells of the reticuloendothelial system. See Vicent and Duncan (2006) Trends Biotechnol 24:39-47 which is incorporated herein by reference in its entirety. The size of the polymer used may be determined by one of skill in the art, but suitably ranges between 5,000 and 40,000 g/mol, suitably between 7,000 and 30,000 g/mol.

Because of the extensive amino acid identity between human IDE and non-human mammalian homologs of human IDE and conservation of the amino acids at the interface between regions corresponding to the IDE-N and IDE-C (FIG. 1, FIG. 5A-D), it is envisioned that, using the guidance provided herein, analogous mutants of homologs of human IDE having altered activity could readily be made and used.

The invention also encompasses polynucleotide sequences encoding the mutant IDE proteins of the invention. The coding sequence may be operably linked to a promoter. The promoter may be a homologous or a heterologous promoter, i.e., a promoter not natively associated with the coding sequence. The promoter may be constitutive or inducible. Suitably, the promoter includes an expression control sequence near the start site of transcription. A promoter may include enhancer or repressor elements that may be non-contiguous with the start site of transcription. The polynucleotide may be provided within a vector, for example, a plasmid, cosmid, or virus.

In another embodiment, the invention provides a cell comprising the polynucleotides described above. The cell is not limited to any particular cell type, but must be capable of expressing the polypeptide encoded by the construct under suitable conditions. Suitable cell types include prokaryotic cells such as bacteria, or eukaryotic cells, including, for example, tumor cells, immortalized cells, primary cells, stem cells, BALB/C cells, neuronal cells, and the like. The polynucleotides may be introduced into cells of a target tissue or into a cell in culture by way of any suitable means. Many such approaches are routinely practiced in the art. For example, one of skill in the art can select any method by which a polynucleotide (e.g., DNA) can be introduced into an organelle, a cell, a tissue or an organism. Cells may be selected to study the effects of IDE activity on specific cell types, or may be selected as a model for diseases that are correlated with altered IDE activity or IDE substrate concentration. Cells used in the assay described in the Examples are also suitable. Suitable methods of administering the construct to a cell may include, but are not limited to, use of non-viral and viral vectors. Suitable viral vectors may include, but are not limited to, retroviruses (including lentiviruses), adenoviruses, adeno-associated viruses and herpes simplex virus type 1 or type 2. In vitro delivery methods include, but are not limited to, transfection, including microinjection, electroporation, calcium phosphate precipitation, using DEAE-dextran followed by polyethylene glycol, direct sonic loading, liposome-mediated transfection and receptor-mediated transfection, microprojectile bombardment, agitation with silicon carbide fibers, desiccation/inhibition-mediated DNA uptake, transduction by viral vector, and/or any combination of such methods.

The following non-limiting Examples are intended to be purely illustrative.

### EXAMPLES

### Protein Preparation and Crystallization

Human insulin, β-Amyloid (1-40), amylin, and glucagon were purchased from RayBiotech, Biosource, Bachem, and Anaspec, respectively. Human IDE-E111Q and selenomethionyl-IDE-E111Q were expressed in E. coli Rosetta(DE3) and B834(DE3)pUBS520 (at 25° C. and 19 hrs IPTG induction), respectively and purified by Ni-NTA, source-Q and superdex S-200 columns. Preformed IDE-substrate complexes isolated from S-200 columns (˜15 mg/ml in buffer [20 mM Tris-HCl, pH 8.0, 50 mM NaCl]) in the presence of a reducing agent, 1 mM Tris-(2-carboxyethyl)-phosphine (TCEP), were mixed with equal volumes of reservoir solution containing 0.1M HEPES (pH 7.0), 12% (w/v) PEGMME-5000, 5% tacsimate and 10% dioxane. Crystals appeared after 1-3 weeks at 18° C. and were then equilibrated in cryoprotective buffer containing well buffer and 30% glycerol. IDE-substrate complex crystals belong to the space group P6₅, with the unit cell dimension a=b=262 Å and c=90 Å, and contain a dimeric IDE-substrate complex per asymmetric unit.

IDE mutants were constructed using the Quik-change kit (Biocrest manufacturing, L.P.) and purified by Ni-NTA and source-Q columns.

### Structure Determination

Data was collected at 14-BM-C and 19-ID stations in the Advanced Photon Source (APS) at Argonne National Laboratory and processed using HKL2000. Anomalous diffraction data were collected on crystals of Se-Met-IDE/insulin B chain complex and 34 of 52 selenium sites were located by the Shake-and-Bake program. Initial phases were obtained by SAD using SHARP (La Fortelle & Bricogne Methods Enzymol 276:472-494 (1997)). DM programs and phase extension were performed on the Zn²⁺-bound IDE-insulin B chain complex. AMoRe was used to obtain the initial phases of structures of Zn²⁺-free IDE in complex with insulin B chain, Aβ, amylin, and glucagon using the template of IDE/insulin B chain. Model building and refinement of IDE-substrate complexes were done using COOT and CNS (Emsley & Cowtan Acta Crystallogr. D 60:2126-2132 (2004)). The final structures of Zn²⁺-IDE-insulin B chain, Zn²⁺-free IDE-insulin B chain, IDE-Aβ, IDE-amylin, and IDE-glucagon had an R_{free} value of 23.3%, 22.5%, 22.3%, 22.5%, 22.5%, and an R_{cryst} value of 20.6%, 20.5%, 20.3%, 19.6%, and 19.8%, respectively. The electron density of the entire IDE dimer (aa 43-1016) is clearly visible, except for a short disordered loop (aa 974-976) and the C-terminal end (aa 1017-1018). Only the structure of Zn²⁺-bound IDE-insulin B chain complex is discussed since the structure of Zn²⁺-free IDE-insulin B chain complex had less clear electron density for the side chains of insulin B chain at the catalytic cleft and crystals of IDE-intact insulin complex did not diffract well.

The structure of IDE-E111Q in complex with insulin B chain is shown in FIG. 3. A representation of the secondary structure of IDE-EB111Q/insulin B chain complex is shown in FIG. 3A, with domains 1, 2, 3, and 4 shown in colored green, blue, yellow, and red, respectively. Zn²⁺ and insulin B chain are colored magenta and orange, respectively. FIG. 3B shows the structure homology of the four domains of IDE. FIG. 3C provides a surface representation of the substrate-binding chamber of IDE. The outer surface of IDE is colored light yellow and the substrate chamber is colored brown. An electrostatic surface representation of the IDE substrate-binding chamber is shown in FIG. 3D. The inner substrate binding chambers of IDE-N and IDE-C are marked by triangles. Negative surface is colored in red, positive in blue, and neutral in white. The catalytic center of IDE is depicted in FIG. 3E. A simulated annealing omit map, colored magenta, is contoured at the 3.5σ level. IDE and insulin B chain are colored cyan and orange, respectively.

### IDE Assay Using Substrate V

Enzyme activity of IDE and IDE mutants were assayed by mixing 100 μl 5 μM fluorogenic peptide substrate V (R&D Systems) at 50 mM potassium phosphate, pH 7.3 and 5 μl IDE proteins at 37° C. for the given time and fluorescence intensity was monitored on a Tecan Safire2 microplate reader at excitation wavelength 327 nm and emission wavelength 395 nm (Li et al. Biochem. Biophys. Res. Commun. 343:1032-1037 (1992)). Indicated quantities of protein (e.g., S132C/E817C and N184C/Q828C) were pre-incubated with 1 mM TCEP or 1 mM K₃Fe(CN)₆ at room temperature for 10 and 60 minutes, respectively to carry out reducing or oxidizing reactions. The fluorogenic substrate was then added and incubated at 37° C. for 30 minutes. To perform the rescue experiment of S132C/E817C and N184C/Q828C by TCEP, the activities of these two mutants in the presence of 1 mM K₃Fe(CN)₆ were first measured after a 30-minute incubation. TCEP was then added to 5 mM and the activities were measured after 30 or 60 minutes for S132C/E817C and N184C/Q828C, respectively.

### Evaluation of Catalytic Activity of IDE Mutants Using Insulin and Amyloid β

The elevated proteolytic activities of the three double cysteine IDE mutants (D426C/K899C, N184C/Q828C, and S132C/E817C) were confirmed when either insulin or Aβ(1-42) were used. To perform the reactions, 10 μL buffer (20 mM HEPES, pH 7.2, 1 mM TCEP) was incubated with 5 μg IDE protein (5 μL of 1 mg/ml protein solution) at room temperature for 5 minutes. The reaction was started by adding 5 μg insulin or 15 μg Aβ(1-42) into the mixture and then incubating for one hour at 37° C. The reaction was stopped by the addition of 5 μl TFA (10%). The 5 μg bacitracin (an inhibitor of wild-type IDE catalytic activity) was then added to serve as a recovery standard of mass spectrometry. The reaction solution (0.5 μL) was mixed with 0.5 μL matrix (α-cyano-4-hydroxycinn) and directly spotted on the metal plate (ABI). For MALDI-TOF, ABI 4700 Maldi TOF/TOF MS was used. The estimated molecular weight of bacitracin, insulin B chain, and Aβ (1-42) were 1,423 daltons, 3,431 daltons, and 4,514 daltons, respectively. The data demonstrated that under identical reaction conditions all three double cysteine IDE mutants degraded both insulin and Aβ(1-42) more effectively than wild-type IDE. This suggested that all three IDE mutants under reduced conditions (with TCEP) were substantially more active than wild-type IDE. This result was consistent with results using fluorogenic substrate V.

The catalytic activity of the D426C/K899C IDE mutant protein was examined by evaluating the kinetic parameters of Aβ degradation using a modification of a fluorescence-based Aβ degradation assay (Leissring, M. A. et al., (2003) J. Biol. Chem. 278: 37314-37320). The assay is based on a derivatized Aβ(1-40) peptide containing fluorescein at the N-terminus and biotin at the C-terminus (FAβB) synthesized by Anaspec (San Jose, Calif.). Hydrolysis of FAβB separates the fluorescent label from the biotin tag. Biotin was attached to the carboxyl-terminal lysine side chain via an aminocaproic acid linker, and 5(6)-carboxyfluorescein (Sigma, St. Louis, Mo., U.S.A.) was attached to the amino terminus via a peptide bond. Aβ(1-40) was synthesized and purified as previously described (see Sciarretta, K. L. et al., (2005) Biochemistry 44: 6003-6014). For kinetic analysis of Aβ degradation by IDE, the fraction of hydrolyzed substrate can be determined by first removing the intact substrate by avidin-agarose precipitation, and then quantifying the remaining fluoresceinated Aβ fragments (see Leissring, M. A. et al., (2003) J. Biol. Chem., 278: 37314-37320). A modification of the published assay was implemented by using unmodified Aβ as the substrate and FAβB as a tracer to monitor degradation, which allowed a better assessment of the kinetics of Aβ degradation (instead of FAβB degradation) by IDE. The Aβ(1-40) concentrations (6.3 to 100 μM) were used in presence of 0.25 μM FAβB. The reaction was performed with IDE in buffer A (50 μL of 50 mM Tris-HCl pH 7.4, 100 mM NaCl, and 0.05% BSA) at 37° C. At the appropriate times, the reaction was stopped by adding 540 μL of buffer A containing 2 mM 1,10-phenanthroline. Neutravidin™-coated agarose (10 μL, Pierce) was added and gently rocked for 30 minutes. The mixture was centrifuged at 14,000×g for 15 minutes, and supernatant solutions were transferred in three 100 μL aliquots to black 96-well plates (Nunc). Fluorescence intensity (λex=488 nm, λem=535 nm) was measured at 37° C. using a Wallac multilabel plate reader (Perkin-Elmer, Waltham, Mass.). The background fluorescence was measured using 0.25 μM FAβB in the absence of enzyme and this signal was subtracted out. The maximum possible fluorescence intensity was determined based on the fluorescence signal from 0.25 μM FAβB and 25 μM Aβ(1-40) reacted with excess D426C/K899C IDE for 30 minutes. It was found that the Kcat of D426C/K899C was 2.5-fold higher than that of wild-type IDE, whereas no significant changes were observed in the values of Km or the Hill coefficient, obtained from two experiments, as shown below in Table 2.

TABLE 2
Kinetic analysis of Aβ degradation by IDE.

| | wild-type IDE | D426C/K899C IDE |
|---|---|---|
| Kcat (sec⁻¹) | 8 ± 1 | 20 ± 2 |
| Km (μM) | 25 ± 4 | 27 ± 7 |
| Hill coefficient | 2.9 ± 0.2 | 2.3 ± 0.3 |

To examine the relative catalytic efficiency of wild-type IDE versus D426C/K899C in a more physiological setting, the ability of these enzymes to degrade Aβ produced naturally by APPswe.3 cells was determined. APPswe.3 cells are an HEK293 cell line that stably expresses myc-epitope tagged human APP-695 harboring the FAD-linked “Swedish” mutation (see Kim, S. H. et al., (2003) J. Biol. Chem., 278: 33992-34002). HEK 293APPswe.3 cells were plated at ˜50% confluency in 60 mm dishes and maintained in 2 mL DMEM supplemented with 1% FBS under normal cell culture conditions for 18 hours. The conditioned medium was collected and centrifuged at 100,000×g for 15 minutes to remove cell debris and membranes, and the supernatant fraction was frozen in aliquots at −20° C. without added protease inhibitors. The conditioned medium of these cells (40 μL), containing abundant Aβ, was incubated with equal amounts of wild-type or mutant IDE. After incubation at 37° C. for different lengths of time, the reactions were quenched with a mixture of 3× Laemmli sample buffer containing 2 mM 1,10-O-phenanthroline. The resulting mixture was boiled and subjected to fractionation by SDS-PAGE (16% Tris-Tricine gels) and Western blot analysis. Substrate-free human insulin degrading enzyme APPsα derivatives and Aβ peptides were detected using the Aβ-specific monoclonal antibody 26D6, which recognizes an epitope between amino acids 1-12 within Aβ (see Kim, S. H. et al., (2004) J. Biol. Chem. 279: 48615-48619). Bound antibodies were visualized by enhanced chemiluminescence (PerkinElmer Life Sciences). For quantification of Western blots, a Bio-Rad XRS Chemidoc imager and Bio-Rad Quantity One software were used. Boltzman fits were determined using Prism software. For incubation times of 10 minutes, wild-type IDE (50 ng) only partially degraded the Aβ present in the conditioned medium; in marked contrast, equal amounts of the IDE-D426C/K899C mutant degraded all of the Aβ in the same time period. The estimated t½ for the degradation of secreted Aβ by wild-type IDE was 11 minutes, while that by the IDE D426C/K899C mutant was 2 minutes. IDE had no effect on the secreted ectodomain of the amyloid precursor protein derivative generated by α-secretase (APPsα) (see Song, E. S. et al., (2005) J. Biol. Chem. 280: 17701-17706), which retains the epitope recognized by the 26D6 antibody and was used as a loading control. These results demonstrate that the D426C/K899C mutation increased the catalytic efficiency of IDE against natural substrates, suggesting that the closed (inactive) state of IDE is the default state in an endogenous context.

While the compositions and methods of this invention have been described in terms of exemplary embodiments, it will be apparent to those skilled in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. In addition, all patents and publications listed or described herein are incorporated in their entirety by reference.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application.

1. A mutant polypeptide of human insulin degrading enzyme having at least one mutation in a region corresponding to human IDE-N or human IDE-C, the mutant having increased activity relative to the activity of insulin degrading enzyme of SEQ ID NO:1.

2. The mutant of claim 1, wherein the mutation is a substitution in an amino acid within five amino acid residues of an amino acid at the interface between IDE-N and IDE-C.

3. The mutant of claim 1, wherein the mutation is a substitution in an amino acid at the interface between IDE-N and IDE-C.

4. The polypeptide of claim 1, wherein at least one amino acid residue of the IDE-N or IDE-C is substituted with a cysteine residue.

5. The polypeptide of claim 1, wherein at least one amino acid residue in each of IDE-N and IDE-C is substituted with a cysteine residue.

6. The polypeptide of claim 5, wherein the substituted cysteine residues are capable of forming a disulfide bond.

7. The polypeptide of claim 1, wherein at least one member of at least one amino acid pair listed in Table 1 is substituted with an amino acid that reduces interactions between the amino acid pair members.

8. The polypeptide of claim 7, wherein the amino acid is substituted with an amino acid selected from the group consisting of alanine, isoleucine, leucine, and glycine.

9. The polypeptide of claim 1, further comprising a chemical modification that increases the stability of the polypeptide.

10. The polypeptide of claim 9, wherein the chemical modification comprises addition of a chemical selected from the group consisting of a polymer and a second polypeptide.

11. The polypeptide of claim 10, wherein the polymer is PEG.

12-19. (canceled)

20. A polynucleotide comprising a sequence encoding the polypeptide of claim 1.

21. The polynucleotide of claim 20, wherein sequence encoding the polypeptide is operably connected to a promoter.

22. A vector comprising the polynucleotide of claim 20.

23. A method of reducing amyloid β or insulin levels in a subject in need thereof, comprising administering to the subject a mutant polypeptide of human insulin degrading enzyme having at least one mutation in a region corresponding to human IDE-N or human IDE-C, the mutant having increased activity relative to the activity of insulin degrading enzyme of SEQ ID NO:1, a polynucleotide encoding the polypeptide, or a vector comprising the polynucleotide encoding the polypeptide in an amount effective to reduce amyloid β or insulin.

24. A composition for reducing amyloid β or insulin levels in a subject in need thereof comprising a pharmaceutically acceptable carrier and at least one polypeptide of claim 1, a polynucleotide encoding the polypeptide, or a vector comprising the polynucleotide.

25-36. (canceled)

37. A method of reducing Aβ comprising contacting a cell expressing Aβ with the polypeptide of claim 1 in an amount effective and under conditions suitable to cleave at least a portion of Aβ.

38. The method of claim 37, wherein contacting comprises expressing a polynucleotide encoding the polypeptide in the cell expressing Aβ or in a second cell.

39. The method of claim 38, wherein the polynucleotide is delivered to the cell by a vector comprising a polynucleotide encoding a mutant polypeptide of human insulin degrading enzyme having at least one mutation in a region corresponding to human IDE-N or human IDE-C, the mutant having increased activity relative to the activity of insulin degrading enzyme of SEQ ID NO:1.

40. The method of claim 37, wherein the Aβ is secreted and contacting occurs extracellularly.

41. A cell comprising the polynucleotide of claim 20.


SEQUENCE LISTING (6 sequences)

SEQ ID NO: 1 - protein, 1020 aa, Homo sapiens
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSPFAYVDPLHCNMAYLYLELLKDSLNEYAYAAELAGLSYDLQNTIYGMYLSVKGTTPMVAQNPRMSLLSSLWLWCLSDTLAEETYNADLAGLKCQLESSPFGVQMRVYGLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIYQIGVQNTYDNAVVGLIDQLIREPAFNTLRTNEALGYIVWTGSRLNCGTVALNVICAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 2 - protein, 989 aa, Drosophila melanogaster
MTIAESSQKSATRKPDSMEPILRLNNIEKSLQDTRDYRGLQLENGLKVLLISDPNTDVSAAALSVQVGHMSDPTNLPGLAHFCEHMLFLGTEKYPHENGYTTYLSQSGGSSNAATYPLMTKYHFHVAPDKLDGALDRFAQFFIAPLFTPSATEREINAVNSEHEKNLPSDLWRIKQVNRHLAKPDHAYSKFGSGNKTTLSEIPKSKNIDVRDELLKFHKQWYSANIMCLAVIGKESLDELEGMVLEKFSEIENKNVKVPGWPRHPYAEERYGQKVKIVPIKDIRSLTISFTTDDLTQFYKSGPDNYLTHLIGHEGKGSILSELRRLGWCNDLMAGHQNTQNGFGFFDIVVDLTQEGLEHVDDIVKIVFQYLEMLRKEGPKKWIFDECVKLNEMRFRFKEKEQPENLVTHAVSSMQIFPLEEVLIAPYLSNEWRPDLIKGLLDELVPSKSRIVIVSQSFEPDCDLAEPYYKTKYGITRVAKDTVQSWENCELNENLKLALPNSFIPTNFDISDVPADAPKHPTIILDTPILRVWHKQDNQFNKPKACMTFDMSNPIAYLDPLNCNLNHMMVMLLKDQLNEYLYDAELASLKLSVMGKSCGIDFTIRGFSDKQVVLLEKLLDHLFDFSIDEKRFDILKEEYVRSLKNFKAEQPYQHSIYYLALLLTENAWANMELLDAMELVTYDRVLNFAKEFFQRLHTECFIFGNVTKQQATDIAGRVNTRLEATNASKLPILARQMLKKREVQLPDRGWFVYQQRNEVHNNCGIEIYLQCGAQTDHTNIMVNLVSQVLSEPCYDCLRTKEQLGYIVFSGVRKVNGANIRIIVQSAKHPSYVEDRIENFLQTYLQVIEDMPLDEFERHKEALAVKKLEKPKTIFQQFSQFYGEIAMQTYHFEREEAEVAILRKISKADFVDYFKKFIAKDGEERRVLSVHIVSQQTDENATSEAEPVEITNMERHKPISDIVTFKSCKELYPIALPFLDIKAKGARSKL

SEQ ID NO: 3 - protein, 998 aa, Danio rerio
MILSTVFGRSIRRVSTLSIRMSDPAVKRVVSDIIRSPEDKREYRGLEFTNGLKAILISDPTTDKSSAALDVHMGSLSDPENISGLAHFCEHMLFLGTEKYPKENEYSQFLSEHAGSSNAFTSGEHTNYYFDVSHEHLQGALDRFAQFFLCPLFDESCKDREVNAVDSEHEKNLMNDAWRLFQLEKATGNPKHPFSKFGTGNKLTLETRPSQQGIDIREELLKFHSTYYSSNLMGLCVLGRETLDELTSMVVKLFGEVENKNVPVPEFPTHPFQEEHLRQFYKVVPIKDIRNLYVTFPIPDLQKYYKSNPGHYLGHLIGHEGPGSLLSELKSKGWVNTLVGGQKEGARGFMFFIINVDLTEEGLLHVEDIIFHMFQYIQKLRTEGPQEWVFQECKDLNTVAFRFKDKERPRGYTSKVAGLLHYYPLEEILAAEYLLEEFRPDLIEMVLDKLRPENVRVAVVSKSFEGQTDRTEEWYGTQYKQEAITDEAIKKWDNADLNGKFKLPMKNEFIPTNFEIYPLEKDSPSAPTLIKDTAMSKVWFKQDDKFFLPKACLNFEFFSPFAYVDPLHCNMAYLYLELLKDSLNEYAYAAELAGLSYDLQNTVYGMYLSVKGYNDKQHILLKKIIEKMATFEIDEKRFDIIKEAYMRSLNNFRAEQPHQHAMYYLRLLMTEVAWTKDELRDALDDVTLPRLKAFIPQLLSRLHIEALLHGNITKQSALEMMQMLEDTLIEHAHTKPLLPSQLIRYREVQVPDGGWYVYQQRNEVHNNCGIEIYYQTDMQNTHENMLLELFCQIISEPCFNTLRTKEQLGYIVFSGPRRANGVQGLRFIIQSEKAPHYLESRVEAFLKTMEKSVEEMGDEAFQKHIQALAIRRLDKPKKLAAECAKYWGEIISQQYNFDRDNIEVAYLKTLTKEHIMQFYRDLLAIDAPRRHKVSVHVLSREMDSCPLVGEFPAQNDVNLAPAPSLPQPSLVQDMTEFKRSLPLFPLTKPHINFMAAKL

SEQ ID NO: 4 - protein, 970 aa, Arabidopsis thaliana
MAVEKSNTTVGGVEILKPRTDNREYRMIVLKNLLQVLLISDPDTDKCAASMSVSVGSFSDPQGLEGLAHFLEHMLFYASEKYPEEDSYSKYITEHGGSTNAYTASEETNYHFDVNADCFDEALDRFAQFFIKPLMSADATMREIKAVDSENQKNLLSDGWRIRQLQKHLSKEDHPYHKFSTGNMDTLHVRPQAKGVDTRSELIKFYEEHYSANIMHLVVYGKESLDKIQDLVERMFQEIQNTNKVVPRFPGQPCTADHLQILVKAIPIKQGHKLGVSWPVTPSIHHYDEAPSQYLGHLIGHEGEGSLFHALKTLGWATGLSAGEGEWTLDYSFFKVSIDLTDAGHEHMQEILGLLFNYIQLLQQTGVCQWIFDELSAICETKFHYQDKIPPMSYIVDIASNMQIYPTKDWLVGSSLPTKFNPAIVQKVVDELSPSNFRIFWESQKFEGQTDKAEPWYNTAYSLEKITSSTIQEWVQSAPDVHLHLPAPNVFIPTDLSLKDADDKETVPVLLRKTPFSRLWYKPDTMFSKPKAYVKMDFNCPLAVSSPDAAVLTDIFTRLLMDYLNEYAYYAQVAGLYYGVSLSDNGFELTLLGYNHKLRILLETVVGKIANFEVKPDRFAVIKETVTKEYQNYKFRQPYHQAMYYCSLLMTEVAWTKDELRDALDDVTLPRLKAFIPQLLSRLHIEALLHGNITKQSALEMMQHIEDVLFNDPKPICRPLFPSQHLTNRVVKLGEGMKYFYHQDGSNPSDENSALVHYIQVHRDDFSMNIKLQLFGLVAKQATFHQLRTVEQLGYITALAQRNDSGIYGVQFIIQSSVKGPGHIDSRVESLLKNFESKLYEMSNEDFKSNVTALIDMKLEKHKNLKEESRFYWREIQSGTLKFNRKEAEVSALKQLQKQELIDFFDEYIKVGAARKKSLSIRVYGSQHLKEMASDKDEVPSPSVEIEDIVGFRKSQPLHGSFRGCGQPKL

SEQ ID NO: 5 - protein, 1027 aa, Saccharomyces cerevisiae
MGVSLLASSSAFVTKPLLTQLVHLSPISLNFTVRRFKPFTCLSRYYTTNPYNMTSNFKTFNLDFLKPDLDERSYRFIELPNKLKALLIQDPKADKAAASLDVNIGAFEDPKNLPGLAHFCEHLLFMGSEKFPDENEYSSYLSKHGGSSNAYTASQNTNYFFEVNHQHLFGALDRFSGFFSCPLFNKDSTDKEINAVNSENKKNLQNDIWRIYQLDKSLTNTKHPYHKFSTGNIETLGTLPKENGLNVRDELLKFHKNFYSANLMKLCILGREDLDTLSDWTYDLFKDVANNGREVPLYAEPIMQPEHLQKIIQVRPVKDLKKLEISFTVPDMEEHWESKPPRILSHLIGHEGSGSLLAHLKKLGWANELSAGGHTVSKGNAFFAVDIDLTDNGLTHYRDVIVLIFQYIEMLKNSLPQKWIFNELQDISNATFKFKQAGSPSSTVSSLAKCLEKDYIPVSRILAMGLLTKYEPDLLTQYTDALVPENSRVTLISRSLETDSAEKWYGTAYKVVDYPADLIKNMKSPGLNPALTLPRPNEFVSTNFKVDKIDGIKPLDEPVLLLSDDVSKLWYKKDDRFWQPRGYIYLSFKLPHTHASIINSMLSTLYTQLANDALKDVQYDAACADLRISFNKTNQGLAITASGFNEKLIILLTRFLQGVNSFEPKKDRFEILKDKTIRHLKNLLYEVPYSQMSNYYNAIINERSWSTAEKLQVFEKLTFEQLINFIPTIYEGVYFETLIHGNIKHEEALEVDSLIKSLIPNNIHNLQVSNNRLRSYLLPKGKTFRYETALKDSQNVNSCIQHVTQLDVYSEDLSALSGLFAQLIHEPCFDTLRTKEQLGYVVFSSSLNNHGTANIRILIQSEHTTPYLEWRINNFYETFGQVLRDMPEEDFEKHKEALCNSLLQKFKNMAEESARYTAAIYLGDYNFTHRQKKAKLVANITKQQMIDFYENYIMSENASKLILHLKSQVENKELNENELDTAKYPTGQLIEDVGAFKSTLFVAPVRQPMKDFEISAPPKLNNSSESE

SEQ ID NO: 6 - protein, 1051 aa, Caenorhabditis elegans
MSLSLLSKFSRGVPSPALAHIAKRTFSSVNFSQILRRPQNSSLRLRLVRNISNSNPLPKMTEAGKNIVLKRHDLIVKGAQDAREYRGLELTNGIRVLLVSDPTTDKSAAALDVKVGHLMDPWELPGLAHFCEHMLFLGTAKYPSENEYSKFLAAHAGSSNAYTSSDHTNYHFDVKPDQLPGALDRFVQFFLSPQFTESATEREVCAVDSEHSNNLNNDLWRFLQVDRSRSKPGHDYGKFGTGNKQTLLEDARKKGIEPRDALLQFHKKWYSSDIMTCCIVGKEPLNVLESYLGTLEFDAIENKKVERKVWEEFPYGPDQLAKRIDVVPIKDTRLVSISFPFPDLNGEFLSQPGHYISHLIGHEGPGSLLSELKRLGWVSSLQSDSHTQAAGFGVYNVTMDLSTEGLEHVDEIIQLMFNYIGMLQSAGPKQWVHDELAELSAVKFRFKDKEQPMTMAINVAASLQYIPFEHILSSRYLLTKYEPERIKELLSMLSPANMQVRVVSQKFKGQEGNTNEPVYGTEMKVTDISPETMKKYENALKTSHHALHLPEKNEYIATNFDQKPRESVKNEHPRLISDDGWSRVWFKQDDEYNMPKQETKLALTTPMVAQNPRMSLLSSLWLWCLSDTLAEETYNADLAGLKCQLESSPFGVQMRVYGYDEKQALFAKHLANRMTNFKIDKTRFDVLFESLKRALTNHAFSQPYLLTQHYNQLLIVDKVWSKEQLLAVCDSVTLEDVQGFAKEMLQAFHMELFVHGNSTEKEAIQLSKELMDVLKSAAPNSRPLYRNEHNPRRELQLNNGDEYVYRHLQKTHDVGCVEVTYQIGVQNTYDNAVVGLIDQLIREPAFNTLRTNEALGYIVWTGSRLNCGTVALNVIVQGPKSVDHVLERIEVFLESVRKEIAEMPQEEFDNQVSGMIARLEEKPKTLSSRFRRFWNEIECRQYNFARREEEVALLKTIKKDDVLELFDKKIRKDAAERRKLAVFVHGKNEDQEAVNTIIKKNAESGKKEKEVLYSDQLRQFLPLYGRPIAAIDLKPIGVDPLEHQETTKSKY